(19) United States
(12) Patent Application Publication
(10) Pub. No.: US 2018/0071349 A1
(43) Pub. Date: Mar. 15, 2018

(54) Herbal Supplement ''Optimal Cholesterol''

(71) Applicant: Zhiting Wang, Westborough, MA (US)

(72) Inventor: Zhiting Wang, Westborough, MA (US)

(21) Appl. No.: 15/261,523

(22) Filed: Sep. 9, 2016

(51) Int. Cl. A61K 36/185; A61K 36/424; A61K 36/725; A61K 36/296; A61K 36/481

(52) U.S. Cl. CPC A61K 36/185; A61K 36/424; A61K 36/725; A61K 2236/39; A61K 36/481; A61K 2236/33; A61K 2236/331; A61K 36/296

(57) ABSTRACT

“Optimal Cholesterol” utilizes edible nature plant extracted by a variety of technology, it is one of the nature plant medicines which can treat atherosclerotic coronary heart disease, cerebral vascular disease and systemic vascular disease in the world. Its function is to soften the arterial blood vessels, and to treat arteriosclerosis, protect and repair the vascular endothelial cells; soft plaque, fibrous plaque and calcified plaque are removed during the process of preventing plaque rupture, bleeding, erosion and ulcer; regulate micro-circulation, enhance the ability of coronary artery blood supply, adjust heart rate, furthermore, there's none toxic side effects after long-term use.

### CROSS-REFERENCE TO RELATED APPLICATIONS

Not Applicable.

### BACKGROUND OF THE INVENTION

Coronary atherosclerotic heart disease (CHD) refers to a heart disease wherein the hardening of coronary arteries causes the vessels to narrow, limiting blood supply and thus causing oxygen deficit in cardiac muscles. It is one of the most common clinical heart diseases. Together with coronary artery function changes, it is referred to as coronary heart disease, or CHD; it is also known as ischemic heart disease (IHD).

CHD is a serious threat to human health. In western developed countries, it accounts for about one third of all deaths in a year, and 50%-75% of deaths caused by heart diseases. According to WHO statistics, CHD is still the most common cause of death in the world, exceeding all deaths caused by tumours combined, and remains top of the list. In recent years, developing countries have witnessed a trend of significant rise in the levels of coronary heart disease; in some developing countries the levels have even surpassed those in developed countries. At the same time, the age of sufferers of coronary heart disease has been getting increasingly younger, the differences in levels between rural and urban areas, men and women, and racial groups have been decreasing.

Treatment methods currently available worldwide are as follows: 1. Drug treatment; 2. Interventional treatment; 3. Surgical treatment; 4. Exercise; 5. Traditional Chinese medicine treatment, and traditional Chinese medicine coupled with Western medicine treatment (in China).

Currently, there is a huge variety of drugs that treat coronary atherosclerotic heart disease in markets worldwide; however, they are by and large Western medicine treatments, whose varieties and scope are mainly of the antianginal and antimyocardial drug type and myocardial infarction and death prevention type, and the categories are as follows:

I. Antianginal and antimyocardial ischemia drugs

1. Nitrate drugs; 2. Beta-blockers; 3. Calcium channel blockers (CCB);

4. Metabolic drugs; 5. Sinus node inhibitors.

II. Myocardial infarction and death prevention drugs

1. Antiplatelet drugs; 2. Lipid-lowering drugs

In the Chinese drug market, while differential treatment of traditional Chinese medicine and Chinese herbology still exists in the process of treating of coronary atherosclerotic heart disease, due to the restrictions of traditional thinking, the treatment has yet to move past theories based on traditional experience: promoting circulation and removing stasis, eliminating stagnation, resuscitating with aromatics, promoting blood circulation by removing obstruction in collaterals, coordinating the spleen and the stomach, and reinforcing the kidney to strengthen the body. These theories have fallen out of touch with the pathogenesis, pathology as well as pathophysiology of CHD that modern medicine has to offer; the formula follows traditional Chinese medicine theories only, without drawing from other sources, causing treatment to focus on temporary relief only, which essentially echoes western medicine; guided by out-dated thinking, selection of drugs tends to be overly conservative; and instead of attempting to improve ancient prescriptions, they continue to be offered without change.

There are close to a hundred different types of traditional Chinese medicine preparations that treat coronary heart disease in Mainland China. Those representing “promoting circulation and removing stasis” include Danshen pian, Fufangdanshen pian, Chuanxiong pian and Naoxintong; preparations representing “resuscitating with aromatics” include Suhexiang wan (Styrax pill), Shexiang Baoxin Wan (Heart-protecting musk pill), Subingdi Wan, Baoxin Wan, and Kuanxiong Wan; drugs designed for “promoting blood circulation by removing obstruction in collaterals” are Tongxinluo and Suxiao Jiuxin Wan among others. These formulae and preparations require long-term use like western medicine CHD treatments, in some cases even throughout a patient's entire lifetime. They also have evident toxic side effects, due to the muscone, artificial calculus bovis, styrax, borneol, bufotoxin, pinellia, flos datura, hornwort herb as well as other traditional Chinese medicine material crude slices that contain heavy metal and toxic substances, and long-term use could lead to a negative effect on liver and kidney functions. Furthermore, these drugs have very limited clinical effects, and they fall in the domain of coronary vasodilator treatment.

### BRIEF SUMMARY OF THE INVENTION

In order to solve the above mentioned issues, the technical solution that the present invention adopted is a functional supplement—Optimal Cholesterol, capable of improving and treating coronary heart disease, myocardial infarction, stenocardia, arrhythmia, tachycardia, bradycardia and cardiac muscle lesions caused by coronary heart disease, characterised by its composition of the following components (as measured per tablet): Rhodiola rosea: 200-400 mg, Seabuckthorn: 250-450 mg, Gynostemma: 250-450 mg, Wild Jujube: 20-60 mg, Epimedium Herb: 30-35 mg, Flatstem Milkvetch Seed: 20-40 mg.

### DETAILED DESCRIPTION OF THE INVENTION

(1) The present invention concerns a new functional supplement (Chinese patent medicine), Optimal Cholesterol, that improves heart functions, belonging to the field of traditional Chinese medicine. It contains the following components (as measured per tablet): Rhodiola rosea: 200-400 mg, Seabuckthorn: 250-450 mg, Gynostemma: 250-450 mg, Wild Jujube: 20-60 mg, Epimedium Herb: 30-35 mg, Flatstem Milkvetch Seed: 20-40 mg.

(2) Preparation method for the new functional supplement:
1. Ethanol extraction: concentration 70-90 W/V, ethanol and crude slices ratio: 8:1. Two 8-hour periods, extraction time for each period 4 hours, temperature 80-100° C.
2. Water extraction: water and crude slices ratio shall be 10:1, two extractions with the first extraction lasting 3 hours and the second extraction lasting 2 hours, temperature 100° C.
3. Water extraction and ethanol precipitation: extract crude slices with water 3 times, combine the extracted fluids, then pressurise and concentrate, adjust concentration to 60-90 W/V, extract and precipitate with ethanol, recycle the ethanol, and then dry and crush.

(3) The present invention uses 6 herbs, all of which are classified as edible and medicinal plants and listed under plants permitted for use in supplements pursuant to provisions of the China Food and Drug Administration. They are safe to consume, effective and free of toxicity and side effects. Multiple chemical components can be obtained through extracting the above 6 herbs with a variety of methods, namely: multiple saponins, multiple flavones, alkaloids, triterpenoids, disaccharide, sterols, amino acids, vitamins A, B1, B2, C and E, dehydroascorbic acid, folic acid, carotene and a variety of trace elements.

(4) Toxic and side effects: none.

(5) The major functions of the invention are as follows:
1. Improving coronary artery vessels, combating myocardial ischemia and improving diastolic function in cardiac muscles;
2. Increasing nutritional blood flow and improves microcirculation in cardiac muscles, lowering myocardial oxygen consumption;
3. Strengthening the heart's contraction and pump function, improving the elasticity of vessels, and lowering resistance surrounding the vessels;
4. Expanding peripheral vessels, decreasing cardiac workload, and increasing coronary flow;
5. Increasing haemoglobin count and blood oxygen content, protecting the cardiac muscles and repairing damaged myocardial cells;
6. Promoting expansion of the L-type calcium channel, increasing calcium concentration in myocardial cells, strengthening the myocardial cells' contraction capability;
7. Binding well with angiotensin II receptor, allowing coronary artery vessels to maintain a good condition for blood supply when stimulated by heat and cold;
8. Protecting the diastolic function of the blood vessel endothelium damaged by lysophosphatidylcholine (LPC);
9. Lowering the total cholesterol (TC), diacylglycerol (TG), low-density lipoprotein cholesterin (LDL-C) and very low-density lipoprotein cholesterin (VLDL-C) in the serum, reducing the oxidative modification of LDL, inhibiting the up-regulated expression of mononuclear cell adhesion molecules, inhibiting inflammatory response, inhibiting the occurrence and development of coronary atherosclerosis, in order to achieve the goals of preventing the formation of thrombus and treating thrombus;
10. Anti-oxidation effect: the chemical components in the present product including beta-carotene, vitamin, folic acid and a variety of trace elements, which can effectively combat the oxidation of free radicals, and reverse the membrane potential functions that have been damaged. The components also have anti-aging and anti-fatigue effects.
11. The total flavonoids component in the product has a significant protective effect on myocardial ischemia, which improves arrhythmia and slowing heart rate caused by blood deficit in cardiac muscles;
12. Protective effect on liver and kidney: the saponin component in the present product can significantly reduce the increased SGDT and SGTP in animals in the CCL4-induced liver damage model, increase the A/G ratio, and reverse the increased AST and ALT in animal livers in the paracetamol model. It can also alleviate proteinuria in the Heymann nephritis rat model, lower blood viscosity, increase oxidation ability and improve kidney functions. In terms of the adenine-feeding induced chronic renal failure and renal tissue fibrosis in rats, not only does it have an anti-fibrosis effect, it can significantly improve kidney functions, where plasma endothelin and tumour necrosis factor show significant improvement and the haemoglobin count demonstrates an evident increase. Compared with the existing medicines in the market for coronary heart disease, “ Optimal Cholesterol” has the following distinctive functions:

1. Give priority to with softening arteries;

2. Prevent thrombus fracture, surface erosion and ulceration;

3. Gradually dissolve thrombus, it is effective for dissolving soft plaque, fibrous plaque and calcified plaque;

4. Regulate micro-circulation, improve myocardial cell oxygen content and pump function;

5. Has obvious therapeutic effect for irregular heartbeat.

So, it can replace a variety of western medicines.

What is claimed is:

1. the combination of rhodiola rosea, seabuckthorn with any two or more than two herb ingredients.
